(19) United States
(12) Patent Application Publication
(10) Pub. No.: US 2023/0136747 A1
(43) Pub. Date: May 4, 2023

(54) METHOD AND SYSTEM TO DETERMINE, TRACK, AND MITIGATE SPECIFIC PHOBIA STIMULATION FOR A PATIENT WITHIN HEALTHCARE SETUP

(71) Applicant: CERNER INNOVATION, INC., Kansas City, KS (US)

(72) Inventors: Preetham Shankar Holalkere, Bengaluru (IN); Pavan Kumar KN, Bengaluru (IN); Shruti Muralidhar Kulkarni, Bengaluru (IN)

(21) Appl. No.: 17/514,165

(22) Filed: Oct. 29, 2021

(51) Int. Cl. G16H 20/70; G16H 10/60; G16H 50/20

(52) U.S. Cl. CPC G16H 20/70; G16H 10/60; G16H 50/20

(57) ABSTRACT

Technologies are provided for determining, tracking, and mitigating specific phobia stimulation for a patient. In embodiments, a patient with specific phobia is automatically and dynamically identified using various clinical data available within the patient record and the exposure rate to stimuli that can potentially trigger phobia in a healthcare setup is mitigated. Initially, data is received from an electronic health care record (EHR) for a patient. The data is analyzed to determine if the patient has a phobia. If it is determined the patient has a phobia that is triggered by a treatment ordered for the patient, mitigations are automatically identified for the treatment ordered for the patient. The mitigations are dynamically provided to the clinician device.

### BACKGROUND

Individuals with phobia can exhibit both fear and/or avoidance of phobia stimulations resulting in either avoidance, rejection, or no show for a healthcare treatment. For those who undergo the treatment, symptoms may include sweating, confusion, disorientation, or panic attacks. For those who choose to cancel at the last minute or simply do not show, valuable resources are wasted and revenue is lost. Currently there are no methods or systems to determine, track, and mitigate specific phobia stimulation for a patient within healthcare setup.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Embodiments of the present invention relate to determining, tracking, and mitigating specific phobia stimulation for a patient. More particularly, embodiments of the present invention automatically and dynamically identify a patient with specific phobia using various clinical data available within the patient record and mitigate the exposure rate to stimuli that can potentially trigger phobia in a healthcare setup. To do so, data is initially received from an electronic health care record (EHR) for a patient. The data is analyzed to determine if the patient has a phobia. If it is determined the patient has a phobia that is triggered by a treatment ordered for the patient, mitigations are automatically identified for the treatment ordered for the patient. The mitigations are dynamically provided to the clinician device.

### BRIEF DESCRIPTION OF THE DRAWINGS

The present invention is described in detail below with reference to the attached drawing figures, wherein:

FIG. 1 is a block diagram of an exemplary computing environment suitable for use in implementing the present disclosure;

FIG. 2 is a block diagram of an exemplary system for determining, tracking, and mitigating specific phobia stimulation for a patient, in accordance with an embodiment of the present disclosure;

FIG. 3 is a block diagram of an exemplary implementation of a phobia tracking engine, in accordance with some embodiments of the present disclosure;

FIGS. 4 and 5 depict illustrative screen displays of the phobia tracking user interface, in accordance with various embodiments of the present disclosure; and

FIG. 6 depicts a flow diagram showing an exemplary method of determining, tracking, and mitigating specific phobia stimulation for a patient, in accordance with various embodiments of the present disclosure.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different components of methods employed, the terms should not be interpreted as implying any particular order among or between various steps herein disclosed unless and except when the order of individual steps is explicitly described.

Recent studies estimate specific phobia affects up to 8-12% of the population at some point during their life span. It is further estimated that specific phobia results in serious impairment for approximately 22%, moderate impairment for approximately 30%, and mild impairment for approximately 48% of adults among the affected population. Some phobias are specific to certain stimulations in healthcare setup. Individuals with phobia can exhibit both fear and/or avoidance of phobia stimulations resulting in either avoidance, rejection, or no show for an advised treatment. For those who undergo the advised treatment with discomfort, symptoms may include sweating, confusion, disorientation, or panic attacks. Additionally, last minute cancellations of surgery and procedures have been reported due to fear, resulting in resource waste and revenue loss for a hospital. Currently there are no methods or systems to determine, track, and mitigate specific phobia stimulation for a patient within healthcare setup.

Embodiments of the present invention relate to determining, tracking, and mitigating specific phobia stimulation for a patient. More particularly, embodiments of the present invention automatically and dynamically identify a patient with specific phobia using various clinical data available within the patient record and mitigate the exposure rate to stimuli that can potentially trigger phobia in a healthcare setup. To do so, data is initially received from an electronic health care record (EHR) for a patient. The data is analyzed to determine if the patient has a phobia. If it is determined the patient has a phobia that is triggered by a treatment ordered for the patient, mitigations are automatically identified for the treatment ordered for the patient. The mitigations are dynamically provided to the clinician device.

In this way, mitigations can be provided during order entry, ward/room/bed/any other resource allocation, treatment plan, care delivery, and the like. These mitigations reduce treatment refusal, denial or last-minute cancellation, and treatment delay which could otherwise adversely affect resources and revenue in a hospital. Moreover, the mitigations reduce patient exposure to stimulant discomfort and contribute to improved patient satisfaction and experience.

Accordingly, in one aspect, an embodiment of the present invention is directed to a method. The method includes receiving, at a phobia tracking engine, data from an electronic health care record for a patient. The method also includes analyzing, at the phobia tracking engine, the data to determine if the patient has a phobia. The method further includes determining, at the phobia tracking engine, the patient has a phobia that is triggered by a treatment ordered for the patient. The method also includes automatically identifying, at the phobia tracking engine, mitigations for the treatment ordered for the patient. The method further includes dynamically providing, at a clinician device, the mitigations for the treatment ordered for the patient.

In another aspect of the invention, an embodiment is directed to one or more computer storage media having computer-executable instructions embodied thereon that, when executed by a computer, causes the computer to perform operations. The operations comprise receiving, at a phobia tracking engine, data from an electronic health care record for a patient. The operations also comprise analyzing, at the phobia tracking engine, the data to determine if the patient has a phobia. The operations further comprise determining, at the phobia tracking engine, the patient has a phobia that is triggered by a treatment ordered for the patient. The operations also comprise automatically identifying, at the phobia tracking engine, mitigations for the treatment ordered for the patient. The operations further comprise dynamically providing, at a clinician device, the mitigations for the treatment ordered for the patient.

In a further aspect, an embodiment is directed to a system that includes one or more processors and a non-transitory computer storage medium storing computer-useable instructions that, when used by the one or more processors, cause the one or more processors to: receive, at an EHR component of a phobia tracking engine, data from an electronic health care record for a patient; analyze, at an analyzing component of the phobia tracking engine, the data to determine if the patient has a phobia; determine, at the analyzing component of the phobia tracking engine, the patient has a phobia that is triggered by a treatment ordered for the patient; automatically identify, at a mitigation component of the phobia tracking engine, mitigations for the treatment ordered for the patient; and dynamically provide, at a clinician device, the mitigations for the treatment ordered for the patient, the mitigations comprising one or more of an alert, a substitute treatment, or a precaution.

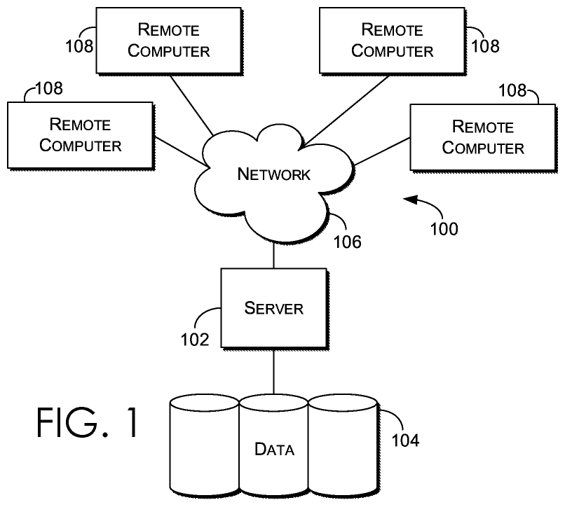

An exemplary computing environment suitable for use in implementing embodiments of the present invention is described below. FIG. 1 is an exemplary computing environment (e.g., medical-information computing-system environment) with which embodiments of the present invention may be implemented. The computing environment is illustrated and designated generally as reference numeral 100. The computing environment 100 is merely an example of one suitable computing environment and is not intended to suggest any limitation as to the scope of use or functionality of the invention. Neither should the computing environment 100 be interpreted as having any dependency or requirement relating to any single component or combination of components illustrated therein.

The present invention might be operational with numerous other purpose computing system environments or configurations. Examples of well-known computing systems, environments, and/or configurations that might be suitable for use with the present invention include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention might be described in the general context of computer-executable instructions, such as program modules, being executed by a computer. Exemplary program modules comprise routines, programs, objects, components, and data structures that perform particular tasks or implement particular abstract data types. The present invention might be practiced in distributed computing environments where tasks are performed by remote processing devices that are linked through a communications network. In a distributed computing environment, program modules might be located in association with local and/or remote computer storage media (e.g., memory storage devices).

With continued reference to FIG. 1, the computing environment 100 comprises a computing device in the form of a control server 102. Exemplary components of the control server 102 comprise a processing unit, internal system memory, and a suitable system bus for coupling various system components, including data store 104, with the control server 102. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 102 typically includes therein, or has access to, a variety of computer-readable media. Computer-readable media can be any available media that might be accessed by control server 102, and includes volatile and nonvolatile media, as well as, removable and nonremovable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by control server 102. Computer storage media does not include signals per se. Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

The control server 102 might operate in a computer network 106 using logical connections to one or more remote computers 108. Remote computers 108 might be located at a variety of locations in a medical or research environment, including clinical laboratories (e.g., molecular diagnostic laboratories), hospitals and other inpatient settings, ambulatory settings, medical billing and financial offices, hospital administration settings, home healthcare environments, clinicians' offices, Center for Disease Control, Centers for Medicare & Medicaid Services, World Health Organization, any governing body either foreign or domestic, Health Information Exchange, and any healthcare/government regulatory bodies not otherwise mentioned. The remote computers 108 might also be physically located in nontraditional medical care environments so that the entire healthcare community might be capable of integration on the network. In various embodiments, the remote computers 108 may represent clients or infrastructure of a client (e.g., devices, applications, services, and the like) or the remote computers 108 may represent user devices corresponding to a support team. The remote computers 108 might be personal computers, servers, routers, network PCs, peer devices, other common network nodes, or the like and might comprise some or all of the elements described above in relation to the control server 102. The devices can be personal digital assistants or other like devices.

Computer networks 106 comprise local area networks (LANs) and/or wide area networks (WANs). Such networking environments are commonplace in offices, enterprise-wide computer networks, intranets, and the Internet. When utilized in a WAN networking environment, the control server 102 might comprise a modem or other means for establishing communications over the WAN, such as the Internet. In a networking environment, program modules or portions thereof might be stored in association with the control server 102, the data store 104, or any of the remote computers 108. For example, various application programs may reside on the memory associated with any one or more of the remote computers 108. It will be appreciated by those of ordinary skill in the art that the network connections shown are exemplary and other means of establishing a communications link between the computers (e.g., control server 102 and remote computers 108) might be utilized.

In operation, an organization might enter commands and information into the control server 102 or convey the commands and information to the control server 102 via one or more of the remote computers 108 through input devices, such as a keyboard, a pointing device (commonly referred to as a mouse), a trackball, or a touch pad. Other input devices comprise microphones, satellite dishes, scanners, or the like. Commands and information might also be sent directly from a remote healthcare device to the control server 102. In addition to a monitor, the control server 102 and/or remote computers 108 might comprise other peripheral output devices, such as speakers and a printer.

Although many other internal components of the control server 102 and the remote computers 108 are not shown, such components and their interconnection are well known. Accordingly, additional details concerning the internal construction of the control server 102 and the remote computers 108 are not further disclosed herein.

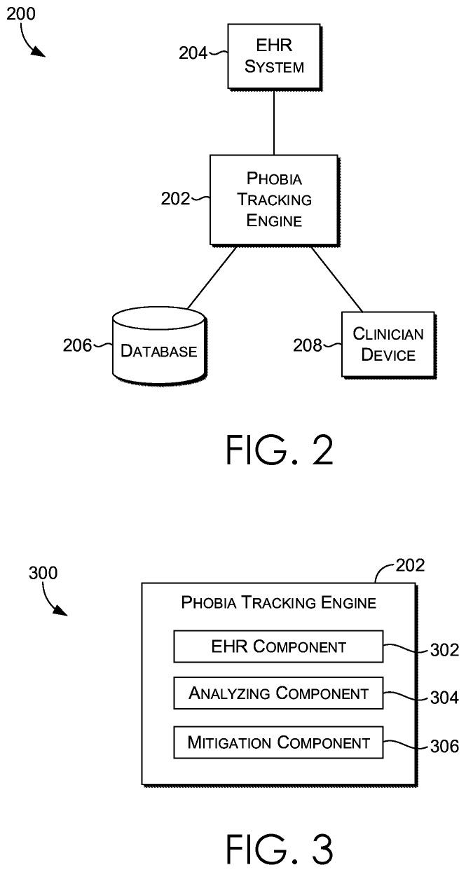

Turning now to FIG. 2, a phobia mitigation system 200 is depicted suitable for use in implementing embodiments of the present invention. The phobia mitigation system 200 is merely an example of one suitable computing system environment and is not intended to suggest any limitation as to the scope of use or functionality of embodiments of the present invention. Neither should the phobia mitigation system 200 be interpreted as having any dependency or requirement related to any single module/component or combination of modules/components illustrated therein.

The phobia mitigation system 200 includes phobia tracking engine 202, EHR system 204, database 206 and clinician device 208, and may be in communication with one another via a network (not shown). The network may include, without limitation, one or more secure local area networks (LANs) or wide area networks (WANs). The network may be a secure network associated with a facility such as a healthcare facility. The secure network may require that a user log in and be authenticated in order to send and/or receive information over the network.

The components/modules illustrated in FIG. 2 are exemplary in nature and in number and should not be construed as limiting. Any number of components/modules may be employed to achieve the desired functionality within the scope of embodiments hereof. Further, components/modules may be located on any number of servers. By way of example only, phobia tracking engine 202 might reside on a server, cluster of servers, or a computing device remote from one or more of the remaining components. Although illustrated as separate systems, the functionality provided by each of these components might be provided as a single component/module. The single unit depictions are meant for clarity, not to limit the scope of embodiments in any form.

Components of the phobia mitigation system 200 may include a processing unit, internal system memory, and a suitable system bus for coupling various system components, including one or more data stores for storing information (e.g., files and metadata associated therewith). Components of the phobia mitigation system 200 typically includes, or has access to, a variety of computer-readable media.

It should be understood that this and other arrangements described herein are set forth only as examples. Other arrangements and elements (e.g., machines, interfaces, functions, orders, and groupings of functions, etc.) can be used in addition to or instead of those shown, and some elements may be omitted altogether. Further, many of the elements described herein are functional entities that may be implemented as discrete or distributed components or in conjunction with other components/modules, and in any suitable combination and location. Various functions described herein as being performed by one or more entities may be carried out by hardware, firmware, and/or software. For instance, various functions may be carried out by a processor executing instructions stored in memory.

Generally, the phobia tracking engine 202 is configured to determine, track, and mitigate specific phobia stimulation for a patient. At patient arrival or as a clinician interacts with a patient chart via clinician device 208, the phobia tracking engine 202 extracts data from the EHR system 204. The data may include problems, diagnoses (e.g., ICD codes), patient history, clinical notes, and/or assessments. Phobia data is extracted by the phobia tracking engine 202 from database 206. For example, the phobia data may include specific phobia pool of data, stimulant pool of data, substitution pool of data, and/or precaution pool of data. The data is compared to phobia data by the phobia tracking engine 202, such as by using a rule engine or pattern matching.

If the phobia tracking engine 202 identifies a match, the patient may have a phobia that is triggered by a treatment ordered for the patient. Accordingly, the phobia tracking engine automatically identifies and dynamically provides, at the clinician device 208, mitigations for the treatment ordered for the patient, the mitigations comprising one or more of an alert, a substitute treatment, or a precaution.

In some embodiments, the phobia tracking engine 202 utilizes clinician feedback (i.e., did the clinician utilize the substitute treatment in accordance with the alert or precaution or did the clinician determine the benefit outweighed the risk?) as part of a feedback loop to punish or reward particular matches in the rule engine. Put another way, the phobia tracking engine 202 compares the frequency of alerts being triggered and the frequency the clinician does not use mitigations or overrides/ignores alerts and automatically makes adjustments to the rule engine by penalizing that particular match. On the other hand, the phobia tracking engine 202 compares the frequency of alerts being triggered and the frequency the clinician uses mitigations and does not override/ignore alerts and automatically makes adjustments to the rule engine by rewarding that particular match.

EHR system 204 is configured to provide information to and store information communicated by, for example, the phobia tracking engine 202 or clinician device 208. The information stored in association with the EHR system 204 may comprise information received from or used by various components of the phobia tracking engine 202 or clinician device 208. Although illustrated as a single system, it is contemplated that multiple EHR systems may be utilized by the present invention. In this way data and context may be aggregated from multiple sources (e.g., EHRs) or multiple locations.

EHR system 204 may include information corresponding to patients associated with one or more healthcare facilities. EHR system 204 may comprise electronic clinical documents such as images, clinical notes, orders, summaries, reports, analyses, information received from the phobia tracking engine 202, clinician device 208, and medical devices (not shown in FIG. 2), or other types of electronic medical documentation relevant to a particular patient's condition and/or treatment. Electronic clinical documents contain various types of information relevant to the condition and/or treatment of a particular patient and can include information relating to, for example, patient identification information, images, alert history, culture results, physical examinations, vital signs, past medical histories, surgical histories, family histories, histories of present illnesses, current and past medications, allergies, symptoms, past orders, completed orders, pending orders, tasks, lab results, other test results, patient encounters and/or visits, immunizations, physician comments, nurse comments, other caretaker comments, clinician assignments, and a host of other relevant clinical information.

The content and volume of such information in the EHR system 204 is not intended to limit the scope of embodiments of the present invention in any way. Further, though EHR system 204 is illustrated as a single, independent component, the EHR system 204 may, in fact, include a plurality of applications and/or storage devices, for instance, a database cluster.

Database 206 generally provides phobia data that is extracted by the phobia tracking engine 202 and compared to patient data extracted from the EHR system 204. The phobia data may include specific phobia pool of data, stimulant pool of data, substitution pool of data, and/or precaution pool of data. Specific phobia pool of data contains phobias attributed to healthcare objects, persons, situations, environments, hospital services, procedures (or treatments), and other care delivery activities. Stimulant pool of data contains stimulants or triggering factors attributed to specific phobias from healthcare objects, persons, situations, environments, hospital services, procedures (or treatments), and other care delivery activities. Substitution pool of data contains alternatives to stimulants to avoid triggering a specific phobia and can be utilized during phobia and stimulant match. Precaution pool of data contains the information to manage the symptoms of phobia responses and can be utilized by the clinician to determine if the benefit outweighs the risk.

Clinician device 208 may be any type of computing device capable of communicating with the phobia tracking engine 208 or interacting with documentation stored in the EHR system 204. Such devices may include any type of mobile and portable devices including cellular telephones, personal digital assistants, tablet PCs, smart phones, and the like.

Referring now to FIG. 3, the phobia tracking engine 202 includes several components and generally provides a decision support system that determines, tracks, and mitigates specific phobia stimulation for a patient. The phobia tracking engine 202 may employ a set of rules to monitor and evaluate a patient in a healthcare setup and trigger alerts, substitutions, and precautions based on care providers actions. In embodiments, the phobia tracking engine 202 may include EHR component 304, analyzing component 306, and mitigation component 308.

EHR component 304 is generally configured to receive data from an EHR for a patient. For example, at patient arrival or as a clinician interacts with a patient chart, EHR component extracts data from the EHR. EHR component 304 may also receive current encounter information, such as from the patient chart as the clinician enters data.

Analyzing component 306 is generally configured to analyze the data received by the EHR component 304 to determine if the patient has a phobia. Moreover, the analyzing component 306 determines if the patient has a phobia that is triggered by a treatment ordered for the patient. Initially, the analyzing component 306 utilizes pattern matching to identify specific data received by EHR component 304 (e.g., admission history, clinical notes, assessment sheets). For example, the data may include items such as “Fear of,” Needle,” “Injection,” “confined space,” “closed space,” “fear of injection,” “fear of needle,” “Trypanophobia,” “Claustrophobia,” and the like. To do so, analyzing component 306 compares terms identified in the data to terms identified in a specific phobia pool of data. The specific phobia pool of data contains phobias attributed to healthcare objects, persons, situations, environments, hospital services, procedures (or treatments), and other care delivery activities.

Analyzing component 306 also utilizes pattern matching to identify potential stimulant matches in a treatment ordered for the patient and a stimulant pool of data. The stimulant pool of data contains stimulants or triggering factors attributed to specific phobias from healthcare objects, persons, situations, environments, hospital services, procedures (or treatments), and other care delivery activities. This enables analyzing component 306 to not only determine the patient has a phobia but that there is also risk of a stimulant in a treatment ordered for the patient triggering the phobia.

Mitigation component 308 is generally configured to identify mitigations for the treatment ordered for the patient. For example, if analyzing component 306 determines the patient has a phobia and the phobia is triggered by a treatment ordered for the patient, mitigation component 308 utilizes pattern matching on a substitution pool of data. The substitution pool of data contains alternatives to stimulants to avoid triggering a specific phobia and can be utilized during phobia and stimulant match. Mitigation component 308 also utilizes pattern matching on a precaution pool of data. Precaution pool of data contains the information to manage the symptoms of phobia if the clinician determines the benefit outweighs the risk and overrides or ignores the alert.

As can be appreciated, each of the components and aspects of the components may operate concurrently to expedite the decision support features. For example, each of the components may perform pattern matching on each pool of data simultaneously or substantially simultaneously (as each phobia is determined). The mitigations for the treatment ordered for the patient are dynamically provided at a clinician device. The mitigations comprises one or more of an alert, a substitute treatment, or a precaution. Feedback based on how the clinician proceeds is provided back to the phobia tracking engine so the rules engine can be dynamically optimized.

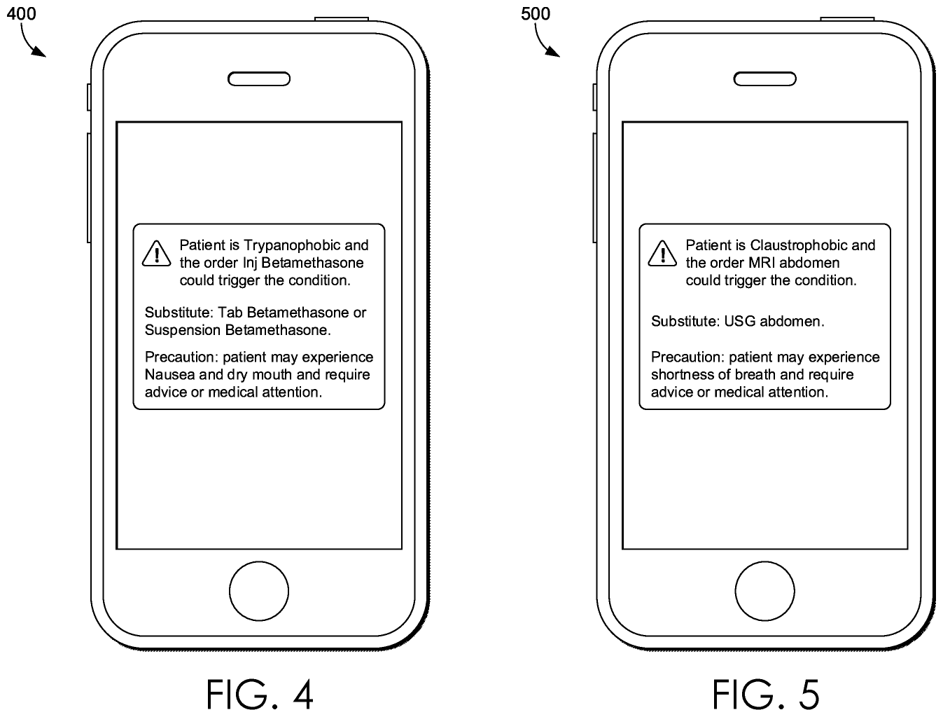

In FIG. 4, an illustrative screen display 400 of the phobia tracking user interface is depicted, in accordance with embodiments of the present invention. In the example shown, a patient with a specific phobia may arrive at a healthcare setup seeking a healthcare service. During the initial assessment, the patient may describe having a history of a fear of needles. The information is recorded by the clinician in the patient chart and captured in an electronic health record of the patient. At a later encounter, the phobia tracking engine analyzes and filters specific phobia related information from the patient chart and compares the data to the specific phobia pool of data in the database. For example, the phobia tracking engine may associate “fear of needles” from the patient chart with “Trypanophobia” in the database.

Additionally, the phobia tracking engine compares the treatment, such as may be described in an order, with the stimulant pool of data in the database to rule out the possibility of exposure of a phobia and a stimulant match. For example, if an order for “1 mg injection Betamethason intramuscular) is signed, the phobia tracking engine finds a match between “Trypanophobia” and “injection Betamethasone.”

In another example, if the provider performs an action that might expose the patient to a phobia stimulation, the phobia tracking engine intelligently tries to mitigate the possible exposure to stimuli. Consider a scenario where a patient has provided a history of missed doses of medications due to “fear of needles.” The patient history may also indicate the patient did not return for an overdue vaccination due to “injection fear.” The patient history may further indicate the patient experienced “dryness of mouth” and “panic attack” while being administered a Dexamethasone injection.

During a current visit, if the provider searches for a particular treatment (e.g., Domperidone), the phobia tracking engine may proactively try and mitigate the exposure to phobia stimuli by providing search results that include “Tab Domperidone” as the first result and “Injection Domperidone” as the last result. In other words, the search results may be ranked based on possible exposure to stimuli. If the provider selects “Injection Domperidone” the phobia tracking engine may prompt the clinician device with an alert indicating “Patient is Trypanophobic and the order ‘Injection Domperidone’ could trigger the condition,” a substitute indicating “‘Tab Domperidone’ or ‘Suspension Domperidone,’” and/or a precaution indicating “Refer to management of panic attack.”

At this point, the phobia tracking engine receives data from the database corresponding to the phobia, the stimulant, an alternative to the stimulant, and precautions. The data is provided to the user interface 400 as illustrated. As shown, user interface 400 is populated with an alert indicating “Patient is Tryanophobic and the order Inj Betamethasone could trigger the condition. Substitute: Tab Betaethasone or Suspension Betamethasone. Precaution: patient may experience Nausea and dry mouth and require advice or medical attention.” The clinician receives the alert via the clinician device and may make a decision based on benefit vs. risk. For example, the clinician may choose a substitution for the stimulant or override the alert and make use of precautions provided by the phobia tracking engine. The phobia tracking engine tracks the decision made by the clinician, such as by tracking changes to the order or cancellations made to the alert and utilizes the feedback to make adjustments to the rules engine (e.g., penalties based on cancellations or rewards based on changes to the order).

Turning to FIG. 5, an illustrative screen display 500 of the phobia tracking user interface is depicted, in accordance with embodiments of the present invention. In the example shown, a patient with a specific phobia may arrive at a healthcare setup seeking a healthcare service. During the initial assessment, the patient may describe having a history of a fear of confined spaces. The information is recorded by the clinician in the patient chart and captured in an electronic health record of the patient. At a later encounter, the phobia tracking engine analyzes and filters specific phobia related information from the patient chart and compares the data to the specific phobia pool of data in the database. For example, the phobia tracking engine may associate “fear of confined spaces” from the patient chart with “Claustrophobia” in the database.

Additionally, the phobia tracking engine compares the treatment, such as may be described in an order, with the stimulant pool of data in the database to rule out the possibility of exposure of a phobia and a stimulant match. For example, if an order for “MRI abdomen” is signed, the phobia tracking engine finds a match between “Claustrophobia” and “MRI abdomen.”

At this point, the phobia tracking engine receives data from the database corresponding to the phobia, the stimulant, an alternative to the stimulant, and precautions. The data is provided to the user interface 500 as illustrated. As shown, user interface 500 is populated with an alert indicating “Patient is Claustrophobic and the order MRI abdomen could trigger the condition. Substitute: USG abdomen. Precaution: patient may experience shortness of breath and require advice or medical attention.” The clinician receives the alert via the clinician device and may make a decision based on benefit vs. risk. For example, the clinician may choose a substitution for the stimulant or override the alert and make use of precautions provided by the phobia tracking engine. The phobia tracking engine tracks the decision made by the clinician, such as by tracking changes to the order or cancellations made to the alert and utilizes the feedback to make adjustments to the rules engine (e.g., penalties based on cancellations or rewards based on changes to the order).

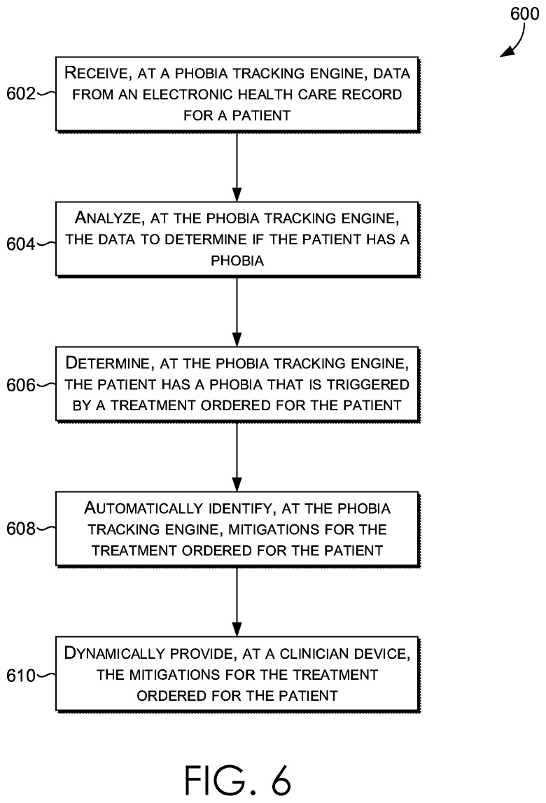

As shown in FIG. 6 a flow diagram is provided illustrating a method 600 for determining, tracking, and mitigating specific phobia stimulation for a patient, in accordance with various embodiments of the present disclosure. Method 600 may be performed by any computing device (such as computing device described with respect to FIG. 1) with access to a phobia mitigation system (such as the one described with respect to FIG. 2) or by one or more components of the phobia mitigation system (such as the phobia tracking engine described with respect to FIGS. 2 and 3). Initially, as shown at step 602, data is received, at a phobia tracking engine, from an electronic health care record for a patient.

At step 604, the data is analyzed, at the phobia tracking engine, to determine if the patient has a phobia. In some embodiments, an order for a treatment for the patient is received. At step 606, it is determined, at the phobia tracking engine, the patient has a phobia that is triggered by a treatment ordered for the patient. The analyzing and/or determining may comprise utilizing a rule engine to perform pattern matching of the data to phobia data in a database. Additionally, a confidence level based on keywords identified during the pattern matching may be attributed. The confidence level may be utilized to determine whether to trigger an alert or may be utilized by the clinician to determine whether to follow a recommendation to utilize an alternative treatment.

At step 608, mitigations for the treatment ordered for the patient are automatically identified, at the phobia tracking engine. At step 610, the mitigations for the treatment ordered for the patient are dynamically provided, at a clinician device. The mitigations may comprise one or more of an alert, a substitute treatment, or a precaution. In embodiments, feedback may be received indicating whether the mitigations were accepted or rejected. The feedback may be utilized to dynamically refining the determining and identifying at the phobia tracking engine. The feedback may also be utilized to dynamically adjust confidence levels.

As can be understood, the present invention provides systems, methods, and user interfaces for providing regulatory document analysis with natural language processing. The present invention has been described in relation to particular embodiments, which are intended in all respects to be illustrative rather than restrictive. Alternative embodiments will become apparent to those of ordinary skill in the art to which the present invention pertains without departing from its scope.

From the foregoing, it will be seen that this invention is one well adapted to attain all the ends and objects set forth above, together with other advantages which are obvious and inherent to the system and method. It will be understood that certain features and subcombinations are of utility and may be employed without reference to other features and subcombinations. This is contemplated and within the scope of the claims.

We claim:

1. A method comprising:
receiving, at a phobia tracking engine, data from an electronic health care record for a patient;
analyzing, at the phobia tracking engine, the data to determine if the patient has a phobia;
determining, at the phobia tracking engine, the patient has a phobia that is triggered by a treatment ordered for the patient;
automatically identifying, at the phobia tracking engine, mitigations for the treatment ordered for the patient; and
dynamically providing, at a clinician device, the mitigations for the treatment ordered for the patient.

2. The method of claim 1, further comprising receiving an order for a treatment for the patient.

3. The method of claim 1, wherein the mitigations comprise one or more of an alert, a substitute treatment, or a precaution.

4. The method of claim 1, wherein the analyzing comprises utilizing a rule engine to perform pattern matching of the data to phobia data in a database.

5. The method of claim 4, further comprising attributing a confidence level based on keywords identified during the pattern matching.

6. The method of claim 1, further comprising receiving feedback indicating whether the mitigations were accepted or rejected.

7. The method of claim 6, further comprising, utilizing the feedback, refining the determining and identifying at the phobia tracking engine.

8. One or more computer storage media having computer-executable instructions embodied thereon that, when executed by a computer, causes the computer to perform operations comprising:
receiving, at a phobia tracking engine, data from an electronic health care record for a patient;
analyzing, at the phobia tracking engine, the data to determine if the patient has a phobia;
determining, at the phobia tracking engine, the patient has a phobia that is triggered by a treatment ordered for the patient;
automatically identifying, at the phobia tracking engine, mitigations for the treatment ordered for the patient; and
dynamically providing, at a clinician device, the mitigations for the treatment ordered for the patient.

9. The media of claim 8, further comprising receiving an order for a treatment for the patient.

10. The media of claim 8, wherein the mitigations comprise one or more of an alert, a substitute treatment, or a precaution.

11. The media of claim 8, wherein the analyzing comprises utilizing a rule engine to perform pattern matching of the data to phobia data in a database.

12. The media of claim 11, further comprising attributing a confidence level based on keywords identified during the pattern matching.

13. The media of claim 8, further comprising receiving feedback indicating whether the mitigations were accepted or rejected.

14. The media of claim 13, further comprising, utilizing the feedback, refining the determining and identifying at the phobia tracking engine.

15. A system comprising:
one or more processors; and
a non-transitory computer storage media storing computer-useable instructions that, when used by the one or more processors, cause the one or more processors to:
receive, at an EHR component of a phobia tracking engine, data from an electronic health care record for a patient;
analyze, at an analyzing component of the phobia tracking engine, the data to determine if the patient has a phobia;
determine, at the analyzing component of the phobia tracking engine, the patient has a phobia that is triggered by a treatment ordered for the patient;
automatically identify, at a mitigation component of the phobia tracking engine, mitigations for the treatment ordered for the patient; and
dynamically provide, at a clinician device, the mitigations for the treatment ordered for the patient, the mitigations comprising one or more of an alert, a substitute treatment, or a precaution.

16. The system of claim 15, further comprising receiving an order for a treatment for the patient.

17. The system of claim 15, wherein the analyzing comprises utilizing a rule engine to perform pattern matching of the data to phobia data in a database.

18. The system of claim 17, further comprising attributing a confidence level based on keywords identified during the pattern matching.

19. The system of claim 15, further comprising receiving feedback indicating whether the mitigations were accepted or rejected.

20. The system of claim 19, further comprising, utilizing the feedback, refining the determining and identifying at the phobia tracking engine.
